(19) United States
(12) Patent Application Publication
(10) Pub. No.: US 2005/0119188 A1
(43) Pub. Date: Jun. 2, 2005

(54) Method of treating breast cancer

(76) Inventors: Brian Long, Scotch Plains, NJ (US); Paul Kirschmeier, Basking Ridge, NJ (US); Walter Robert Bishop, Pompton Plains, NJ (US); Leila Alland, Chatham Township, NJ (US); Craig Tendler, Teaneck, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 10/980,915

(22) Filed: Nov. 4, 2004

Related U.S. Application Data
(60) Provisional application No. 60/517,820, filed on Nov. 6, 2003.

(51) Int. Cl. A61K 38/09; A61K 31/473; A61K 31/453; A61K 31/137; A61K 31/56

(52) U.S. Cl. 514/17; 514/290; 514/171; 514/649; 514/320

(57) ABSTRACT

A method of treating breast cancer is disclosed. The method comprises administering an FTI, at least one antihormonal agent (e.g., an aromatase inhibitor, an antiestrogen, and an LHRH analogue), optional chemotherapeutic agents (e.g., Trastuzumab), and optional radiation. For example, the treatment of breast cancer using the FTI and Anastrozole is disclosed. Also disclosed is a method of treating breast cancer using the FTI Anastrozole and Fulvestrant. Also disclosed are pharmaceutical compositions comprising an FTI, at least one antihormonal agent and a pharmaceutically acceptable carrier; and compositions comprising an FTI, at least one antihormonal agent, at least one chemotherapeutic agent, and a pharmaceutically acceptable carrier; and compositions comprising an FTI, at least one chemotherapeutic agent, and a pharmaceutically acceptable carrier.

### REFERENCE TO RELATED APPLICATION

This application claims the benefit of U.S. Provisional Application Ser. No. 60/517,820 filed on Nov. 6, 2003, the disclosure of which is incorporated herein by reference thereto.

### BACKGROUND

The treatment and prevention of Breast Cancer is of significant interest to those skilled in the art. Therefore, treatments for and preventatives for Breast Cancer would be a welcome contribution to the art. This invention provides such a contribution.

### SUMMARY OF THE INVENTION

This invention provides methods of treating breast cancer (i.e., postmenopausal and premenopausal breast cancer, e.g., hormone-dependent breast cancer) in a patient in need of such treatment wherein said treatment comprises the administration of a farnesyl transferase inhibitor (FTI) with hormonal therapies (i.e., antihormonal agents).

The methods of this invention include the treatment of hormone-dependent metastatic and advanced breast cancer, adjuvant therapy for hormone-dependent primary and early breast cancer, the treatment of ductal carcinoma in situ, and the treatment of inflammatory breast cancer in situ.

Optionally, neoadjuvant therapy (i.e., the use of chemotherapeutic agents) is used in combination with the FTI and hormonal therapies in the methods of this invention.

Optionally, radiation treatment can be administered in the methods of this invention.

The methods of this invention can also be used to prevent breast cancer in patients having a high risk of developing breast cancer.

The FTI is

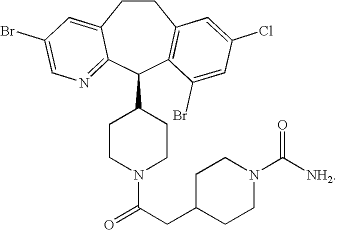

### BRIEF DESCRIPTION OF THE DRAWINGS

FIG. 1 shows the inhibition of estrogen-stimulated MCF-7 cell growth by the combination of FTI plus 4-OH Tamoxifen.

FIG. 2 shows the inhibition of estrogen-stimulated MCF-7 cell growth by the combination of FTI plus Fulvestrant.

FIG. 3 shows the inhibition of MCF-7arom breast tumor by the combination of FTI (20 mpk, b.i.d.) plus Anastrozole (5.0 mpk, b.i.d.).

FIG. 4 shows the inhibition of MCF-7arom breast tumor by the combination of FTI (40 mpk, b.i.d.) plus Anastrozole (5.0 mpk, b.i.d.).

FIG. 5 shows inhibition of MCF-7arom breast tumor by the combination of FTI (60 mpk, b.i.d.) plus Anastrozole (5.0 mpk, b.i.d.).

FIG. 6 shows the final volumes of MCF-7arom breast tumors after 28 days of treatment with the combination of FTI plus Anastrozole.

FIG. 7 shows the inhibition of MCF-7arom breast tumor by the combination of FTI (40 mpk, b.i.d.) plus Letrozole (2.5 mpk, q.d.).

FIG. 8 shows the inhibition of MCF-7arom breast tumor by the combination of FTI (40 mpk, b.i.d.) plus Tamoxifen (25 mpk, q.d.).

FIG. 9 shows the inhibition of MCF-7 breast tumor by the combination of FTI (40 mpk, b.i.d.) plus Tamoxifen (25 mpk, q.d.).

### DETAILED DESCRIPTION OF THE INVENTION

As used herein the following terms have the following meanings unless otherwise defined.
“At least one”—means one or more than one, e.g., 1, 2 or 3, or 1 or 2, or 1.
“Consecutively”—means one following the other.
“Concurrently”—means at the same time.
“i.m.”—means intramuscularly.
“mpk”—means milligrams per kilogram (of body weight)
“Patient”—means a mammal, and preferably means a human.
“p.o.”—means by mouth, i.e., orally.
“s.c.”—means subcutaneoulsy.
“Therapeutically effective amount” or “effective amount”—means the amount needed to obtain the desired therapeutic effect, e.g., the amount needed to provide a complete response, the amount needed to inhibit or stop tumor growth, reduce tumor size, cause tumor regression, alleviate or cause the disappearance of one or more symptoms caused by the cancer, eliminate the tumor, and/or provide long-term disease stabilization (growth arrest) of the tumor
“LHRH”—represents Luteinizing Hormone Releasing Hormone.

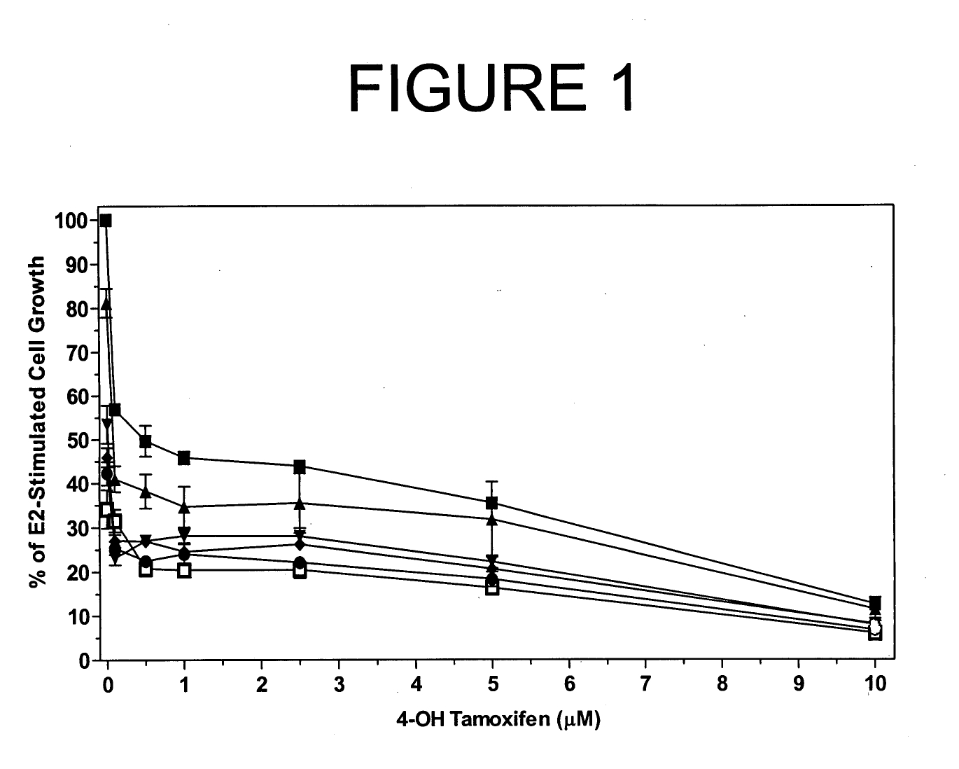

In FIG. 1:
▪ represents 0.0 μM of FTI
▴ represents 0.01 μM of FTI
▾ represents 0.05 μM of FTI
♦ represents 0.10 μM of FTI
● represents 0.50 μM of FTI
□ represents 1.0 μM of FTI
   FIG. 1 shows the inhibition of estrogen-stimulated MCF-7 cell growth by the combination of FTI plus 4-OH Tamoxifen. At each of the concentrations of single-agent 4-OH Tamoxifen tested, the combination of FTI plus 4-OH Tamoxifen was more effective at inhibiting MCF-7 cell proliferation.

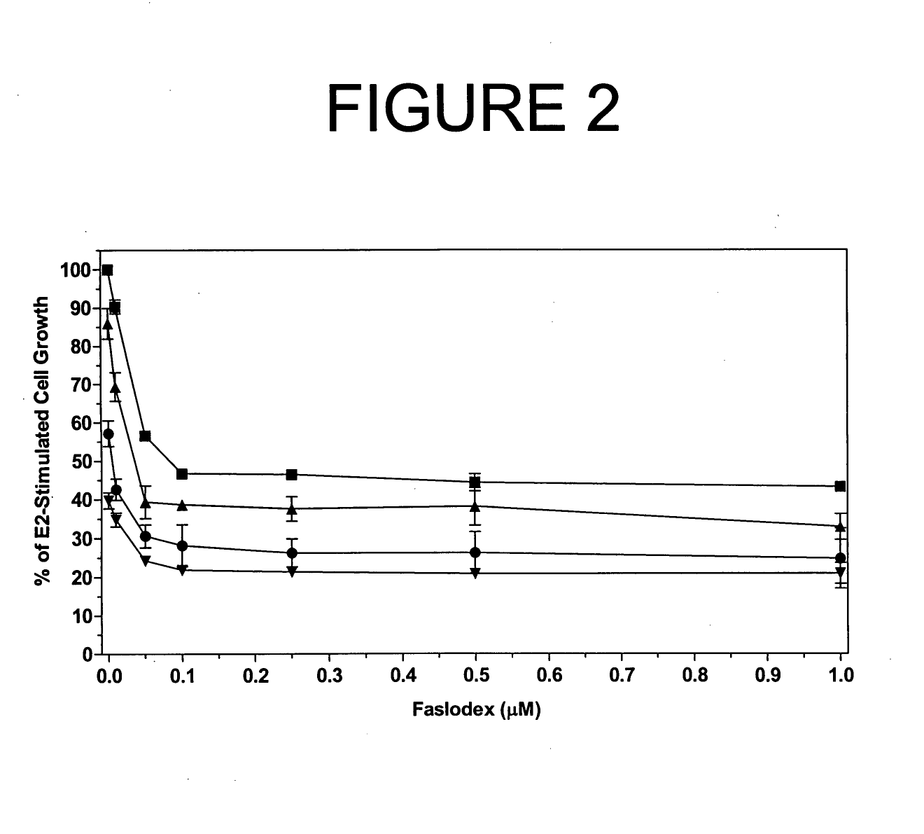

In FIG. 2:
▪ represents 0.0 μM of FTI
▴ represents 0.01 μM of FTI
● represents 0.10 μM of FTI
▾ represents 1.0 μM of FTI
   FIG. 2 shows the inhibition of estrogen-stimulated MCF-7 cell growth by the combination of FTI plus Fulvestrant. At each of the concentrations of single-agent Fulvestrant tested, the combination of FTI plus Fulvestrant was more effective at inhibiting MCF-7 cell proliferation.

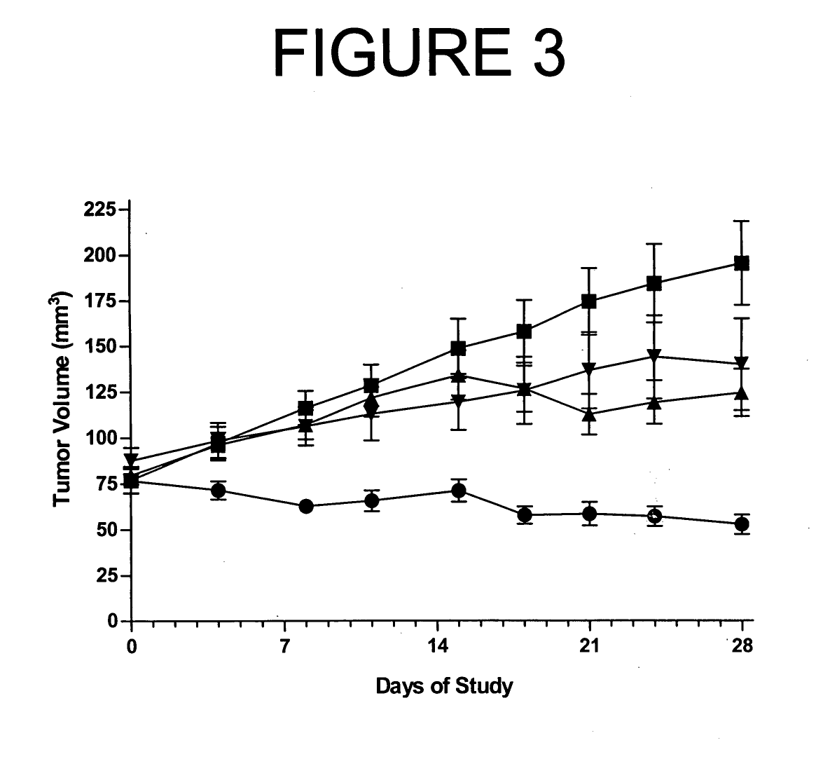

In FIG. 3:
represents Vehicle
A represents Anastrozole (5 mpk)
V represents FTI (20 mpk)
represents FTI+Anastrozole
   FIG. 3 shows the inhibition of MCF-7arom breast tumor by the combination of FTI (20 mpk, b.i.d.) plus Anastrozole (5.0 mpk, b.i.d.). Treatment with single-agent FTI and Anastrozole inhibited the growth of MCF-7arom human breast tumors. The combination of FTI plus Anastrozole was more effective at inhibiting tumor growth and induced tumor regression.

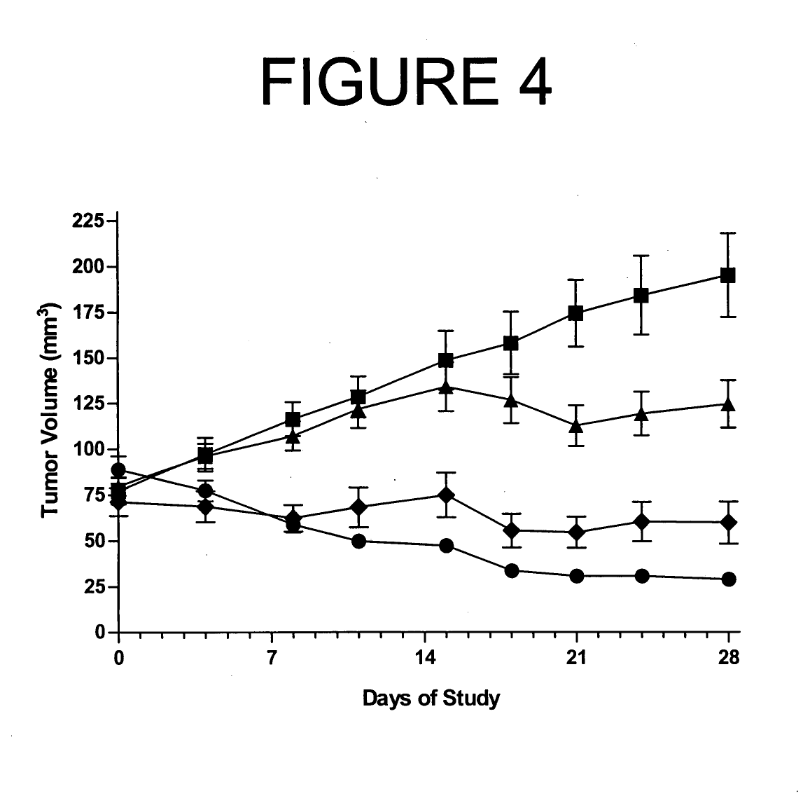

In FIG. 4:
represents Vehicle
A represents Anastrozole (5 mpk)
represents FTI (40 mpk)
represents FTI+Anastrozole
   FIG. 4 shows the inhibition of MCF-7arom breast tumor by the combination of FTI (40 mpk, b.i.d.) plus Anastrozole (5.0 mpk, b.i.d.). Treatment with single-agent FTI and Anastrozole inhibited the growth of MCF-7arom human breast tumors. The combination of FTI plus Anastrozole was more effective at inhibiting tumor growth and induced tumor regression.

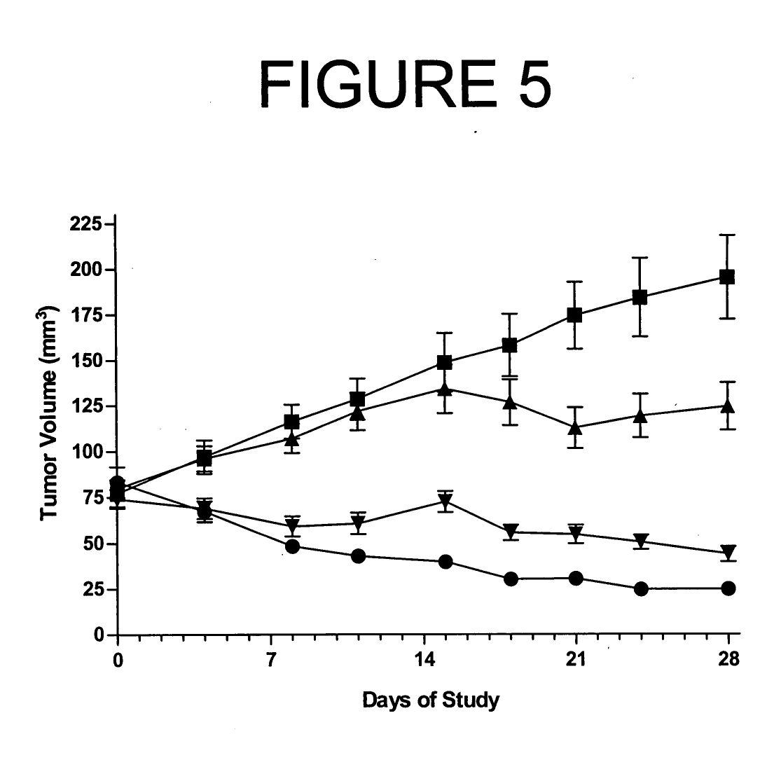

In FIG. 5:
▪ represents Vehicle
▴ represents Anastrozole (5 mpk)
▾ represents FTI (60 mpk)
● represents FTI+Anastrozole
   FIG. 5 shows the inhibition of MCF-7arom breast tumor by the combination of FTI (60 mpk, b.i.d.) plus Anastrozole (5.0 mpk, b.i.d.). Treatment with single-agent FTI and Anastrozole inhibited the growth of MCF-7arom human breast tumors. The combination of FTI plus Anastrozole was more effective at inhibiting tumor growth and induced tumor regression.

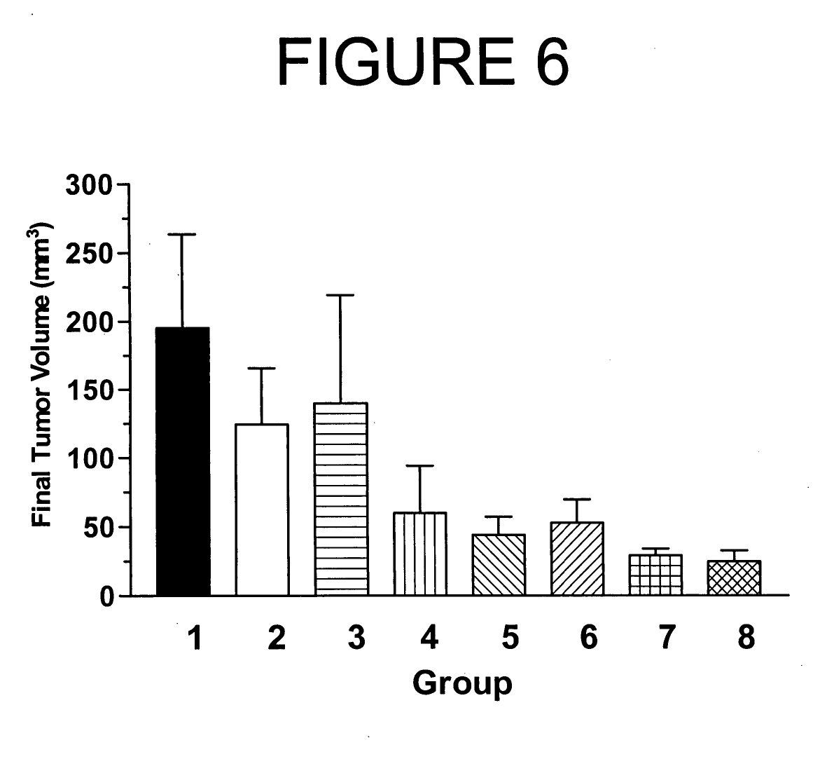

In FIG. 6:
Group 1 represents Vehicle
Group 2 represents Anastrozole (5 mpk)
Group 3 represents FTI (20 mpk)
Group 4 represents FTI (40 mpk)
Group 5 represents FTI (60 mpk)
Group 6 represents FTI (20 mpk)+Anastrozole (5 mpk)
Group 7 represents FTI (40 mpk)+Anastrozole (5 mpk)
Group 8 represents FTI (60 mpk)+Anastrozole (5 mpk)
   FIG. 6 shows the final volumes of MCF-7arom breast tumors after 28 days of treatment with the combination of FTI plus Anastrozole. Treatment with the combination of FTI plus Anastrozole was superior to treatment with either single-agent FTI and Anastrozole. Moreover, each of the combination treatments induced marked tumor regression.

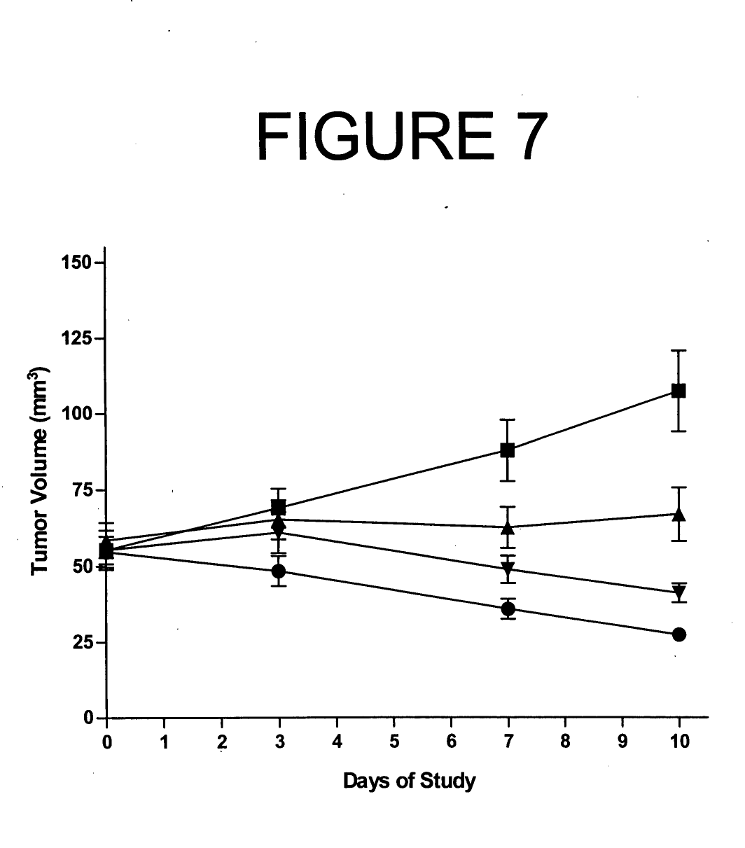

In FIG. 7:
▪ represents Vehicle
▾ represents Letrozole (2.5 mpk)
▴ represents FTI (40 mpk)
● represents Letrozole+FTI
   FIG. 7 shows the inhibition of MCF-7arom breast tumor by the combination of FTI (40 mpk, b.i.d.) plus Letrozole (2.5 mpk, q.d.). Treatment with single-agent FTI and Letrozole inhibited the growth of MCF-7arom human breast tumors. The combination of FTI plus Letrozole was more effective at inhibiting tumor growth and induced tumor regression.

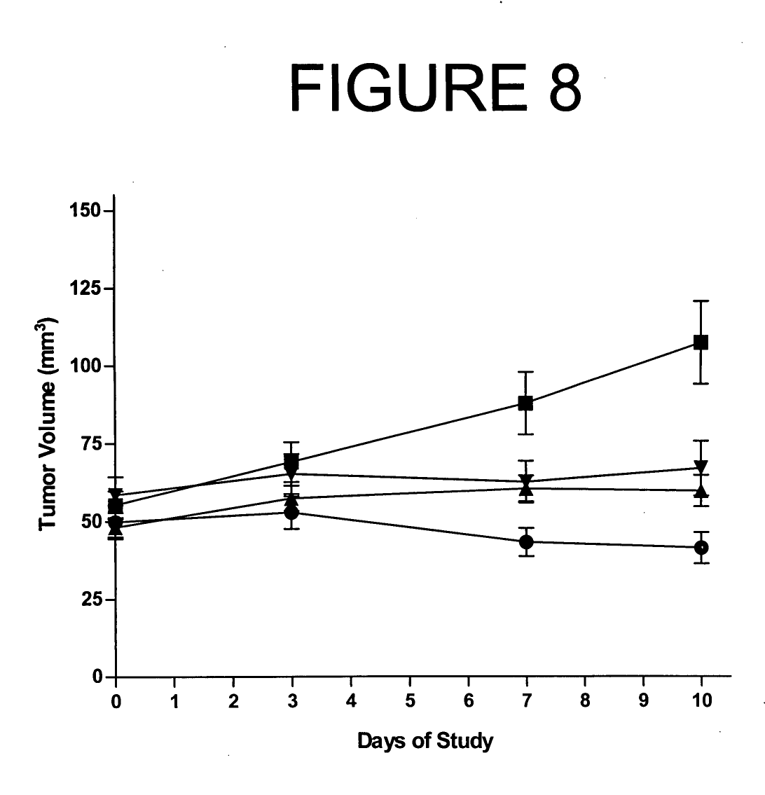

In FIG. 8:
Vehicle
▴ represents Tamoxifen (25 mpk)
▾ represents FTI (40 mpk)
● represents Tamoxifen+FTI
   FIG. 8 shows the inhibition of MCF-7arom breast tumor by the combination of FTI (40 mpk, b.i.d.) plus Tamoxifen (25 mpk, q.d.). Treatment with single-agent FTI and Tamoxifen inhibited the growth of MCF-7arom human breast tumors. The combination of FTI plus Tamoxifen was more effective at inhibiting tumor growth and induced tumor regression.

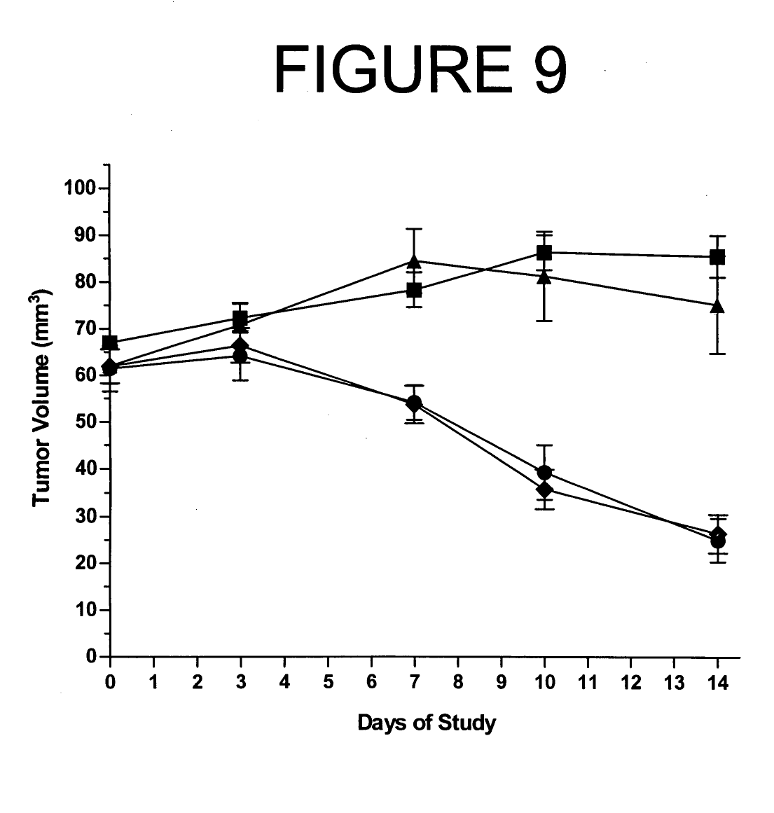

In FIG. 9:
▪ represents Vehicle
▴ represents Tamoxifen (25 mpk)+
♦ represents FTI (40 mpk)
● represents Tamoxifen+FTI
   FIG. 9 shows the inhibition of MCF-7 breast tumor by the combination of FTI (40 mpk, b.i.d.) plus Tamoxifen (25 mpk, q.d.). Treatment with single-agent Tamoxifen inhibited MCF-7 breast tumor growth and single-agent FTI induced tumor regression. The combination of FTI plus Tamoxifen also induced tumor regression.

The methods of this invention are directed to the use of a combination of FTI and drugs for the treatment of breast cancer, i.e., this invention is directed to a combination therapy for the treatment of breast cancer. Those skilled in the art will appreciate that the FTI and drugs are generally administered as individual pharmaceutical compositions. The use of a pharmaceutical composition comprising more than one drug is within the scope of this invention.

The FTI, also referred to as a farnesyl protein transferase (FPT) inhibitor, can also be represented as 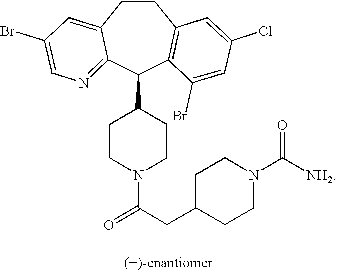
This FTI is available from Schering Corporation, Kenilworth, N.J. See also, U.S. Pat. No. 5,874,442, U.S. Pat. No. 6,632,455B2 and U.S. 2004/0122232 (published Jun. 24, 2004), the disclosures of each being incorporated herein by reference thereto.

The FTI used in the methods described herein also include the use of a pharmaceutical composition comprising the FTI. Such a composition would be available from Schering Corporation under the tradename Sarasar.

Thus, this invention is directed to a method of treating (or preventing) breast cancer (i.e., postmenopausal and premenopausal breast cancer, e.g., hormone-dependent breast cancer) in a patient in need of such treatment comprising administering to said patient a therapeutically effective amount of the farnesyl transferase inhibitor: 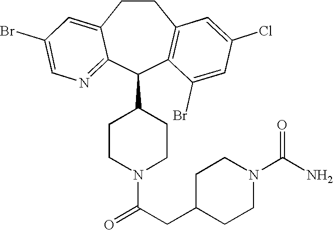
and a therapeutically effective amount of at least one antihormonal agent selected from the group consisting of:
(a) aromatase inhibitors;
(b) antiestrogens; and
(c) LHRH analogues; and
said treatment optionally including the administration of at least one chemotherapeutic agent.

The FTI is preferably administered orally, and is most preferably administered in capsule form.

Examples of aromatase inhibitors include but are not limited to: Anastrozole (e.g., Arimidex), Letrozole (e.g., Femara), Exemestane (Aromasin), Fadrozole and Formestane (e.g., Lentaron).

Examples of antiestrogens include but are not limited to: Tamoxifen (e.g., Nolvadex), Fulvestrant (e.g., Faslodex), Raloxifene (e.g., Evista), and Acolbifene.

Examples of LHRH analogues include but are not limited to: Goserelin (e.g., Zoladex) and Leuprolide (e.g., Leuprolide Acetate, such as Lupron or Lupron Depot).

Examples of chemotherapeutic agents include but are not limited to: Trastuzumab (e.g., Herceptin), Gefitinib (e.g., Iressa), Erlotinib (e.g., Erlotinib HCl, such as Tarceva), Bevacizumab (e.g., Avastin), Cetuximab (e.g., Erbitux), and Bortezomib (e.g., Velcade).

Preferably, when more than one antihormonal agent is used, each agent is selected from a different category of agent. For example, one agent is an aromatase inhibitor (e.g., Anastrozole, Letrozole, or Exemestane) and one agent is an antiestrogen (e.g., Tamoxifen or Fulvestrant).

One embodiment of this invention is directed to a method of treating or preventing breast cancer in a patient in need of such treatment wherein said treatment comprises administering a therapeutically effective amount of the FTI and at least one antihormonal agent selected from the group consisting of:
(a) aromatase inhibitors;
(b) antiestrogens; and
(c) LHRH analogues; and
   administering an effective amount of at least one chemotherapeutic agent

Another embodiment of this invention is directed to a method of treating or preventing breast cancer in a patient in need of such treatment wherein said treatment comprises administering a therapeutically effective amount of the FTI and at least one antihormonal agent selected from the group consisting of:
(a) aromatase inhibitors;
(b) antiestrogens; and
(c) LHRH analogues.

Another embodiment of this invention is directed to a method of treating or preventing breast cancer in a patient in need of such treatment wherein said treatment comprises administering a therapeutically effective amount of the FTI and at least one antihormonal agent selected from the group consisting of:
(a) aromatase inhibitors; and
(b) antiestrogens.

Another embodiment of this invention is directed to a method of treating or preventing breast cancer in a patient in need of such treatment wherein said treatment comprises administering a therapeutically effective amount of the FTI, at least one antihormonal agent selected from the group consisting of:
(a) aromatase inhibitors; and
(b) antiestrogens; and
   at least one chemotherapeutic agent.

Another embodiment of this invention is directed to a method of treating or preventing breast cancer in a patient in need of such treatment wherein said treatment comprises administering a therapeutically effective amount of the FTI and at least one aromatase inhibitor.

Another embodiment of this invention is directed to a method of treating or preventing breast cancer in a patient in need of such treatment wherein said treatment comprises administering a therapeutically effective amount of the FTI, at least one aromatase inhibitor, and at least one chemotherapeutic agent.

Another embodiment of this invention is directed to a method of treating or preventing breast cancer in a patient in need of such treatment wherein said treatment comprises administering a therapeutically effective amount of:
the FTI;
at least one antihormonal agent selected from the group consisting of:
   (a) aromatase inhibitors that are selected from the group consisting of Anastrozole, Letrozole, Exemestane, Fadrozole and Formestane;
   (b) antiestrogens that are selected from the group consisting of: Tamoxifen, Fulvestrant, Raloxifene, and Acolbifene; and
   (c) LHRH analogues that are selected from the group consisting of: Goserelin and Leuprolide; and
administering an effective amount of at least one chemotherapeutic agents are selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of:
the FTI;
at least one antihormonal agent selected from the group consisting of:
   (a) aromatase inhibitors that are selected from the group consisting of Anastrozole, Letrozole, Exemestane, Fadrozole and Formestane;
   (b) antiestrogens that are selected from the group consisting of: Tamoxifen, Fulvestrant, Raloxifene, and Acolbifene; and
   (c) LHRH analogues that are selected from the group consisting of: Goserelin and Leuprolide.

Another embodiment of this invention is directed to a method of treating or preventing breast cancer in a patient in need of such treatment wherein said treatment comprises administering a therapeutically effective amount of:
the FTI;
at least one antihormonal agent selected from the group consisting of:
   (a) aromatase inhibitors that are selected from the group consisting of Anastrozole, Letrozole, Exemestane, Fadrozole and Formestane; and
   (b) antiestrogens that are selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of:
the FTI;
at least one antihormonal agent selected from the group consisting of:
   (a) aromatase inhibitors that are selected from the group consisting of Anastrozole, Letrozole, Exemestane, Fadrozole and Formestane;
   (b) antiestrogens that are selected from the group consisting of: Tamoxifen, Fulvestrant, Raloxifene, and Acolbifene; and
administering an effective amount of at least one chemotherapeutic agents are selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of:
the FTI; and
at least one aromatase inhibitor selected from the group consisting of Anastrozole, Letrozole, Exemestane, Fadrozole and Formestane.

Another embodiment of this invention is directed to a method of treating or preventing breast cancer in a patient in need of such treatment wherein said treatment comprises administering a therapeutically effective amount of:
the FTI;
at least one aromatase inhibitor that is selected from the group consisting of Anastrozole, Letrozole, Exemestane, Fadrozole and Formestane; and
administering an effective amount of at least one chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of:
(a) the FTI
(b) at least one aromatase inhibitor; and
(c) at least one LHRH analogue.

Another embodiment of this invention is directed to a method of treating or preventing breast cancer in a patient in need of such treatment wherein said treatment comprises administering a therapeutically effective amount of:
(a) the FTI
(b) at least one antiestrogen; and
(c) at least one LHRH analogue.

Another embodiment of this invention is directed to a method of treating or preventing breast cancer in a patient in need of such treatment wherein said treatment comprises administering a therapeutically effective amount of:
(a) the FTI
(b) at least one aromatase inhibitor that is selected from the group consisting of Anastrozole, Letrozole, Exemestane, Fadrozole and Formestane; and
(c) at least one LHRH analogue that is selected from the group consisting of: Goserelin and Leuprolide.

Another embodiment of this invention is directed to a method of treating or preventing breast cancer in a patient in need of such treatment wherein said treatment comprises administering a therapeutically effective amount of:
(a) the FTI
(b) at least one antiestrogen that is selected from the group consisting of: Tamoxifen, Fulvestrant, Raloxifene, and Acolbifene; and
(c) at least one LHRH analogue that is selected from the group consisting of: Goserelin and Leuprolide.

Another embodiment of this invention is directed to a method of treating or preventing breast cancer in a patient in need of such treatment wherein said treatment comprises administering a therapeutically effective amount of the FTI and Anastrozole.

Another embodiment of this invention is directed to a method of treating or preventing breast cancer in a patient in need of such treatment wherein said treatment comprises administering a therapeutically effective amount of the FTI and Letrazole.

Another embodiment of this invention is directed to a method of treating or preventing breast cancer in a patient in need of such treatment wherein said treatment comprises administering a therapeutically effective amount of the FTI and Exemestane.

Another embodiment of this invention is directed to a method of treating or preventing breast cancer in a patient in need of such treatment wherein said treatment comprises administering a therapeutically effective amount of the FTI and and Fadrozole.

Another embodiment of this invention is directed to a method of treating or preventing breast cancer in a patient in need of such treatment wherein said treatment comprises administering a therapeutically effective amount of the FTI and Formestane.

Another embodiment of this invention is directed to a method of treating or preventing breast cancer in a patient in need of such treatment wherein said treatment comprises administering a therapeutically effective amount of the FTI and Tamoxifen.

Another embodiment of this invention is directed to a method of treating or preventing breast cancer in a patient in need of such treatment wherein said treatment comprises administering a therapeutically effective amount of the FTI Fulvestrant.

Another embodiment of this invention is directed to a method of treating or preventing breast cancer in a patient in need of such treatment wherein said treatment comprises administering a therapeutically effective amount of the FTI and Raloxifene.

Another embodiment of this invention is directed to a method of treating or preventing breast cancer in a patient in need of such treatment wherein said treatment comprises administering a therapeutically effective amount of the FTI and Acolbifene.

Another embodiment of this invention is directed to a method of treating or preventing breast cancer in a patient in need of such treatment wherein said treatment comprises administering a therapeutically effective amount of the FTI and Goserelin.

Another embodiment of this invention is directed to a method of treating or preventing breast cancer in a patient in need of such treatment wherein said treatment comprises administering a therapeutically effective amount of the FTI and and Leuprolide.

Another embodiment of this invention is directed to a method of treating or preventing breast cancer in a patient in need of such treatment wherein said treatment comprises administering a therapeutically effective amount of the FTI, Anastrozole, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of the FTI, Letrozole, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of the FTI, Exemestane, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of the FTI, Fadrozole, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of the FTI, Formestane, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of the FTI, Anastrozole, and Tamoxifen.

Another embodiment of this invention is directed to a method of treating or preventing breast cancer in a patient in need of such treatment wherein said treatment comprises administering a therapeutically effective amount of the FTI, Letrozole, and Tamoxifen.

Another embodiment of this invention is directed to a method of treating or preventing breast cancer in a patient in need of such treatment wherein said treatment comprises administering a therapeutically effective amount of the FTI, Exemestane, and Tamoxifen.

Another embodiment of this invention is directed to a method of treating or preventing breast cancer in a patient in need of such treatment wherein said treatment comprises administering a therapeutically effective amount of the FTI, Fadrozole, and Tamoxifen.

Another embodiment of this invention is directed to a method of treating or preventing breast cancer in a patient in need of such treatment wherein said treatment comprises administering a therapeutically effective amount of the FTI, Formestane, and Tamoxifen.

Another embodiment of this invention is directed to a method of treating or preventing breast cancer in a patient in need of such treatment wherein said treatment comprises administering a therapeutically effective amount of the FTI, Anastrozole, and Fulvestrant.

Another embodiment of this invention is directed to a method of treating or preventing breast cancer in a patient in need of such treatment wherein said treatment comprises administering a therapeutically effective amount of the FTI, Letrozole, and Fulvestrant.

Another embodiment of this invention is directed to a method of treating or preventing breast cancer in a patient in need of such treatment wherein said treatment comprises administering a therapeutically effective amount of the FTI, Exemestane, and Fulvestrant.

Another embodiment of this invention is directed to a method of treating or preventing breast cancer in a patient in need of such treatment wherein said treatment comprises administering a therapeutically effective amount of the FTI, Fadrozole, and Fulvestrant.

Another embodiment of this invention is directed to a method of treating or preventing breast cancer in a patient in need of such treatment wherein said treatment comprises administering a therapeutically effective amount of the FTI, Formestane, and Fulvestrant.

Another embodiment of this invention is directed to a method of treating or preventing breast cancer in a patient in need of such treatment wherein said treatment comprises administering a therapeutically effective amount of the FTI, Anastrozole, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Letrozole, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Exemestane, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Fadrozole, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Formestane, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Raloxifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Goserelin, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Leuprolein, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Anastrozole, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Letrozole, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Exemestane, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Fadrozole, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Formestane, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Anastrozole, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Letrozole, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Exemestane, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Fadrozole, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Formestane, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Anastrozole, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Letrozole, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Exemestane, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Fadrozole, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Formestane, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of the FTI, Goserelin and Tamoxifen.

Another embodiment of this invention is directed to a method of treating or preventing breast cancer in a patient in need of such treatment wherein said treatment comprises administering a therapeutically effective amount of the FTI, Goserelin, and Fulvestrant.

Another embodiment of this invention is directed to a method of treating or preventing breast cancer in a patient in need of such treatment wherein said treatment comprises administering a therapeutically effective amount of the FTI, Goserelin, and Raloxifene.

Another embodiment of this invention is directed to a method of treating or preventing breast cancer in a patient in need of such treatment wherein said treatment comprises administering a therapeutically effective amount of the FTI, Goserelin and Acolbifene.

Another embodiment of this invention is directed to a method of treating or preventing breast cancer in a patient in need of such treatment wherein said treatment comprises administering a therapeutically effective amount of the FTI, Leuprolide, and Tamoxifen.

Another embodiment of this invention is directed to a method of treating or preventing breast cancer in a patient in need of such treatment wherein said treatment comprises administering a therapeutically effective amount of the FTI, Leuprolide, and Fulvestrant.

Another embodiment of this invention is directed to a method of treating or preventing breast cancer in a patient in need of such treatment wherein said treatment comprises administering a therapeutically effective amount of the FTI, Leuprolide, and Raloxifene.

Another embodiment of this invention is directed to a method of treating or preventing breast cancer in a patient in need of such treatment wherein said treatment comprises administering a therapeutically effective amount of the FTI, Leuprolide and Acolbifene.

Another embodiment of this invention is directed to a method of treating or preventing breast cancer in a patient in need of such treatment wherein said treatment comprises administering a therapeutically effective amount of the FTI, Goserelin and Anastrozole.

Another embodiment of this invention is directed to a method of treating or preventing breast cancer in a patient in need of such treatment wherein said treatment comprises administering a therapeutically effective amount of the FTI, Goserelin and Letrozole.

Another embodiment of this invention is directed to a method of treating or preventing breast cancer in a patient in need of such treatment wherein said treatment comprises administering a therapeutically effective amount of the FTI, Goserelin and Exemestane.

Another embodiment of this invention is directed to a method of treating or preventing breast cancer in a patient in need of such treatment wherein said treatment comprises administering a therapeutically effective amount of the FTI, Goserelin and Fadrozole.

Another embodiment of this invention is directed to a method of treating or preventing breast cancer in a patient in need of such treatment wherein said treatment comprises administering a therapeutically effective amount of the FTI, Goserelin and Formestane.

Another embodiment of this invention is directed to a method of treating or preventing breast cancer in a patient in need of such treatment wherein said treatment comprises administering a therapeutically effective amount of the FTI, Leuprolide and Anastrozole.

Another embodiment of this invention is directed to a method of treating or preventing breast cancer in a patient in need of such treatment wherein said treatment comprises administering a therapeutically effective amount of the FTI, Leuprolide and Letrozole.

Another embodiment of this invention is directed to a method of treating or preventing breast cancer in a patient in need of such treatment wherein said treatment comprises administering a therapeutically effective amount of the FTI, Leuprolide and Exemestane.

Another embodiment of this invention is directed to a method of treating or preventing breast cancer in a patient in need of such treatment wherein said treatment comprises administering a therapeutically effective amount of the FTI, Leuprolide and Fadrozole.

Another embodiment of this invention is directed to a method of treating or preventing breast cancer in a patient in need of such treatment wherein said treatment comprises administering a therapeutically effective amount of the FTI, Leuprolide and Formestane.

A preferred embodiment of this invention is directed to the treatment or prevention of breast cancer in a patient in need of such treatment, said treatment comprising the administration of a therapeutically effective amount of the FTI and Anastrozole.

Another preferred embodiment of this invention is directed to the treatment or prevention of breast cancer in a patient in need of such treatment, said treatment comprising the administration of a therapeutically effective amount of the FTI and Letrozole.

Another preferred embodiment of this invention is directed to the treatment or prevention of breast cancer in a patient in need of such treatment, said treatment comprising the administration of a therapeutically effective amount of the FTI and Exemestane.

Another preferred embodiment of this invention is directed to the treatment or prevention of breast cancer in a patient in need of such treatment, said treatment comprising the administration of a therapeutically effective amount of the FTI and Tamoxifen.

Another preferred embodiment of this invention is directed to the treatment or prevention of breast cancer in a patient in need of such treatment, said treatment comprising the administration of a therapeutically effective amount of the FTI and Fulvestrant.

Another preferred embodiment of this invention is directed to the treatment or prevention of breast cancer in a patient in need of such treatment, said treatment comprising the administration of a therapeutically effective amount of the FTI, Anastrozole, and Fulvestrant.

Another embodiment of this invention is directed to the treatment or prevention of breast cancer in a patient in need of such treatment, said treatment comprising the administration of a therapeutically effective amount of the FTI, Letrozole, and Fulvestrant.

Another embodiment of this invention is directed to the treatment or prevention of breast cancer in a patient in need of such treatment, said treatment comprising the administration of a therapeutically effective amount of the FTI, Exemestane, and Fulvestrant.

Another embodiment of this invention is directed to the treatment or prevention of breast cancer in a patient in need of such treatment, said treatment comprising the administration of a therapeutically effective amount of the FTI, Anastrozole, and Tamoxifen.

Another embodiment of this invention is directed to the treatment or prevention of breast cancer in a patient in need of such treatment, said treatment comprising the administration of a therapeutically effective amount of the FTI, Letrozole, and Tamoxifen.

Another embodiment of this invention is directed to the treatment or prevention of breast cancer in a patient in need of such treatment, said treatment comprising the administration of a therapeutically effective amount of the FTI, Exemestane, and Tamoxifen.

Other embodiments of this invention are directed to any of the above described embodiments wherein the chemotherapeutic agent is Trastuzumab.

Other embodiments of this invention are directed to any of the above described embodiments wherein the method is directed to a method of treating breast cancer.

The FTI inhibitor, antihormonal agents and chemotherapeutic agents can be administered concurrently or sequentially.

The antihormonal agents and optional chemotherapeutic agents are administered according to their protocols, dosage amounts, and dosage forms that are well know to those skilled in the art (e.g., the Physician's Desk Reference or published literature). For example, for Tamoxifen, Fulvestrant, Raloxifene, Anastrozole, Letrozole, Exemestane, Leuprolide and Goserelin, see the Physician's Desk Reference, 57^{th} Edition, 2003, published by Thomas PDR at Montvale, N.J. 07645-1742, the disclosure of which is incorporated herein by reference thereto.

In general, in the methods of this invention:
the FTI can be administered daily (e.g., once per day, and preferably twice a day),
the aromatase inhibitors can be administered in accordance with the known protocol for the aromatase inhibitor used (e.g., once per day),
the antiestrogens can be administered in accordance with the known protocol for the antiestrogen used (e.g., from once a day to once a month),
the LHRH analogue can be administered in accordance with the known protocol for the LHRH analogue used (e.g., once a month to once every three months), and
the chemotherapeutic agent can be administered in accordance with the known protocol for the chemotherapeutic agent used (e.g., from once a day to once a week).

Radiation therapy, if administered, is generally administered according to known protocols before administration of the FTI, antihormonal agents and optional chemotherapeutic agents.

Treatment according to the methods of this invention is continuous (i.e., a continuous dosing schedule is followed). The treatment is continued until there is a complete response, or until the skilled clinician determines that the patient is not benefiting from the treatment (for example, when there is disease progression).

The continuous treatment protocol can be changed to a discontinuous treatment schedule if, in the judgment of the skilled clinician, the patient would benefit from a discontinuous treatment schedule with one or more of the administered drugs. For example, the FTI can be given using a discontinous treatment schedule while the remaining drugs used in the treatment are given as described herein. An example of a discontinuous treatment protocol for the FTI is a repeating cycle of three weeks with the FTI followed by one week without the FTI.

After a complete response is achieved, maintenance therapy with the FTI can be continued using the dosing described in the methods of this invention. Maintenance therapy can also include administration of the antihormonal agents using the dosing described in the methods of this invention. Maintenance therapy can just be with the antihormonal agents. For example, after a complete response is achieved, an aromatase inhibitor (e.g., Anastrozole, Letrozole or Exemestane) can be continued for up to five years. Or, for example, an antiestrogen, e.g., Tamoxifen, may be used for up to five years after a complete response is achieved. Or, for example, an antiestrogen (e.g., Tamoxifen) can be used for up to five years after a complete response is achieved followed by the use of an aromatase inhibitor (e.g., Anastrozole, Letrozole or Exemestane) for up to five years.

The FTI is administered continuously in a total daily dose of about 100 mg to about 600 mg. Usually this amount is administered in divided doses, with twice a day being preferred. Most preferably the FTI is dosed twice a day in an amount of about 50 mg to about 300 mg per dose. More preferably the FTI is dosed twice a day in an amount of about 100 mg to about 200 mg per dose. Examples include the FTI being dosed twice a day at 100 mg per dose. Examples also include the FTI being dosed twice a day at 200 mg per dose.

Anastrozole is administered p.o. and is dosed once a day in amounts of about 0.5 to about 10 mg per dose, and preferably in an amount of about 1.0 mg per dose.

Letrozole is administered p.o. and is dosed once a day in amounts of about 1.0 to about 10 mg per dose, and preferably in an amount of about 2.5 mg per dose.

Exemestane is administered p.o. and is dosed once a day in amounts of about 10 to about 50 mg per dose, and preferably in an amount of about 25 mg per dose.

Fadrozole is administered p.o. and is dosed twice a day in amounts of about 0.5 to about 10 mg per dose, and preferably in an amount of about 2.0 mg per dose.

Formestane is administered i.m. and is dosed once every two weeks in amounts of about 100 to about 500 mg per dose, and preferably in an amount of about 250 mg per dose.

Tamoxifen is administered p.o. and is dosed once a day in amounts of about 10 to about 100 mg per dose, and preferably in an amount of about 20 mg per dose.

Fulvestrant is administered i.m. and is dosed once a month in amounts of about 100 to about 1000 mg per dose, and preferably in an amount of about 250 mg per dose.

Raloxifene is administered p.o. and is dosed once a day in amounts of about 10 to about 120 mg per dose, and preferably in an amount of about 60 mg per dose.

Acolbifene is administered p.o. and is dosed once a day in amounts of about 5 to about 20 mg per dose, and preferably in an amount of about 20 mg per dose.

Goserelin is administered s.c. and is dosed once a month, or once every three months, in amounts of about 2 to about 20 mg per dose, and preferably in an amount of about 3.6 mg per dose when administered once a month, and preferably in an amount of about 10.8 mg per dose when administered once every three months.

Leuprolide is administered s.c. and is dosed once a month, or once every three months, in amounts of about 2 to about 20 mg per dose, and preferably in an amount of about 3.75 mg per dose when administered once a month, and preferably in an amount of about 11.25 mg per dose when administered once every three months.

Trastuzumab is administered by i.v. and is dosed once a week in amounts of about 2 to about 20 mpk per dose, and preferably in an amount of about 2 mpk per dose. Trastuzumab is generally initially administered in a loading dose that is generally twice the dose of the weekly dose. Thus, for example, a 4 mpk loading dose is administered and then dosing is 2 mpk per dose per week.

Gefitinib is administered p.o. and is dosed once a day in amounts of about 100 to about 1000 mg per dose, and preferably in an amount of about 250 mg per dose.

Erlotinib is administered p.o. and is dosed once a day in amounts of about 100 to about 500 mg per dose, and preferably in an amount of about 150 mg per dose.

Bevacizumab is administered i.v. and is dosed once every two weeks in amounts of about 2.5 to about 15 mg per kilogram of body weight per dose, and preferably in an amount of about 10 mg per kilogram per dose.

Cetuximab is administered i.v. and is dosed once a week in amounts of about 200 to about 500 mg per meter squared dose, and preferably in an amount of about 250 mg per meter squared per dose.

Bortezomib is administered i.v. and is dosed twice a week for 2 weeks followed by a 10 day rest period (21 day treatment cycle) for a maximum of 8 treatment cycles in amounts of about 1.0 to about 2.5 mg per meter squared per dose, and preferably in an amount of about 1.3 mg per meter squared per dose.

In a preferred embodiment of this invention breast cancer is treated (or prevented) in a patient in need of such treatment wherein said treatment comprises administering to said patient:
the FTI orally in an amount of about 50 mg to about 300 mg per dose wherein each dose is administered twice a day, and
Anastrozole p.o. in an amount of about 0.5 to about 10 mg per dose wherein each dose is given once a day.

In most preferred embodiment of this invention breast cancer is treated (or prevented) in a patient in need of such treatment wherein said treatment comprises administering to said patient:
the FTI orally in an amount of about 100 to 200 mg per dose, wherein each dose is administered twice a day, and
Anastrozole in an amount of about 1.0 mg per dose wherein each dose is given once a day.

In another embodiment of this invention breast cancer is treated (or prevented) in a patient in need of such treatment wherein said treatment comprises administering to said patient:
the FTI orally in an amount of about 50 mg to about 300 mg per dose wherein each dose is administered twice a day, and
Letrozole p.o. in an amount of about 1.0 to about 10 mg per dose wherein each dose is given once a day.

In another embodiment of this invention breast cancer is treated (or prevented) in a patient in need of such treatment wherein said treatment comprises administering to said patient:
the FTI in an amount of about 100 to 200 mg per dose, wherein each dose is administered twice a day, and
Letrozole p.o. in an amount of about 2.5 mg per dose wherein each dose is given once a day.

In another embodiment of this invention breast cancer is treated (or prevented) in a patient in need of such treatment wherein said treatment comprises administering to said patient:
the FTI orally in an amount of about 50 mg to about 300 mg per dose wherein each dose is administered twice a day, and
Exemestane p.o. in an amount of about 10 to about 50 mg per dose wherein each dose is given once a day.

In another embodiment of this invention breast cancer is treated (or prevented) in a patient in need of such treatment wherein said treatment comprises administering to said patient:
the FTI in an amount of about 100 to 200 mg per dose, wherein each dose is administered twice a day, and
Exemestane in an amount of about 25 mg per dose wherein each dose is given once a day.

In another embodiment of this invention breast cancer is treated (or prevented) in a patient in need of such treatment wherein said treatment comprises administering to said patient:
the FTI orally in an amount of about 50 mg to about 300 mg per dose wherein each dose is administered twice a day, and
Fulvestrant i.m. in an amount of about 100 to about 1000 mg per dose wherein each dose is given once a month.

In another embodiment of this invention breast cancer is treated (or prevented) in a patient in need of such treatment wherein said treatment comprises administering to said patient:
the FTI orally in an amount of about 100 to 200 mg per dose, wherein each dose is administered twice a day, and
Fulvestrant i.m. in an amount of about 250 mg per dose wherein each dose is given once a month.

In another embodiment of this invention breast cancer is treated (or prevented) in a patient in need of such treatment wherein said treatment comprises administering to said patient:
the FTI p.o. in an amount of about 50 mg to about 300 mg per dose wherein each dose is administered twice a day, and
Tamoxifen p.o. in an amount of about 10 to about 100 mg per dose wherein each dose is given once a day.

In another embodiment of this invention breast cancer is treated (or prevented) in a patient in need of such treatment wherein said treatment comprises administering to said patient:
the FTIp.o. in an amount of about 100 to 200 mg per dose, wherein each dose is administered twice a day, and
Tamoxifen p.o. in an amount of about 20 mg per dose wherein each dose is given once a day.

In other embodiments of the invention breast cancer is treated in a patient in need of such treatment wherein said treatment comprises the administration of the FTI, one of the aromatase inhibitors (e.g., Anastrozole, Letrozole, or Exemestane, and preferably Anastrozole), and one of the antiestrogens (e.g., Fulvestrant or Tamoxifen), wherein the FTI, aromatase inhibitor and antiestrogen are administered in the dosages described above.

Thus, for example in another embodiment of this invention breast cancer is treated (or prevented) in a patient in need of such treatment wherein said treatment comprises administering to said patient of:
the FTI p.o. in an amount of about 50 mg to about 300 mg per dose wherein each dose is administered twice a day,
Anastrozole p.o. in an amount of about 0.5 to about 10 mg per dose wherein each dose is given once a day, and
Fulvestrant i.m. in an amount of about 100 to about 1000 mg per dose wherein each dose is given once a month.

In another embodiment of this invention breast cancer is treated (or prevented) in a patient in need of such treatment wherein said treatment comprises administering to said patient:
the FTI p.o in an amount of about 100 to 200 mg per dose, wherein each dose is administered twice a day,
Anastrozole p.o. in an amount of about 1.0 mg per dose wherein each dose is given once a day, and
Fulvestrant i.m. in an amount of about 250 mg per dose wherein each dose is given once a month.

In another embodiment of this invention breast cancer is treated (or prevented) in a patient in need of such treatment wherein said treatment comprises administering to said patient:
the FTI p.o. in an amount of about 50 mg to about 300 mg per dose wherein each dose is administered twice a day,
Letrozole p.o in an amount of about 1.0 to about 10 mg per dose wherein each dose is given once a day, and
Fulvestrant in an amount of about 100 to about 1000 mg per dose wherein each dose is given once a month.

In another embodiment of this invention breast cancer is treated (or prevented) in a patient in need of such treatment wherein said treatment comprises administering to said patient:
the FTI p.o. in an amount of about 100 to 200 mg per dose, wherein each dose is administered twice a day,
Letrozole p.o. in an amount of about 2.5 mg per dose wherein each dose is given once a day, and
Fulvestrant i.m. in an amount of about 250 mg per dose wherein each dose is given once a month.

In another embodiment of this invention breast cancer is treated (or prevented) in a patient in need of such treatment wherein said treatment comprises administering to said patient:
the FTI p.o. in an amount of about 50 mg to about 300 mg per dose wherein each dose is administered twice a day,
Exemestane p.o. in an amount of about 10 to about 50 mg per dose wherein each dose is given once a day, and
Fulvestrant i.m. in an amount of about 100 to about 1000 mg per dose wherein each dose is given once a month.

In another embodiment of this invention breast cancer is treated (or prevented) in a patient in need of such treatment wherein said treatment comprises administering to said patient:
the FTI p.o. in an amount of about 100 to 200 mg per dose, wherein each dose is administered twice a day,
Exemestane p.o. in an amount of about 25 mg per dose wherein each dose is given once a day, and
Fulvestrant i.m. in an amount of about 250 mg per dose wherein each dose is given once a month.

In another embodiment of this invention breast cancer is treated (or prevented) in a patient in need of such treatment wherein said treatment comprises administering to said patient:
the FTI p.o. in an amount of about 50 mg to about 300 mg per dose wherein each dose is administered twice a day,
Anastrozole p.o. in an amount of about 0.5 to about 10 mg per dose wherein each dose is given once a day, and
Tamoxifen p.o.in an amount of about 10 to about 100 mg per dose wherein each dose is given once a day.

In another embodiment of this invention breast cancer is treated (or prevented) in a patient in need of such treatment wherein said treatment comprises administering to said patient:
the FTI p.o. in an amount of about 100 to 200 mg per dose, wherein each dose is administered twice a day,
Anastrozole p.o. in an amount of about 1.0 mg per dose wherein each dose is given once a day, and
Tamoxifen p.o. in an amount of about 20 mg per dose wherein each dose is given once a day.

In another embodiment of this invention breast cancer is treated (or prevented) in a patient in need of such treatment wherein said treatment comprises administering to said patient:
the FTI p.o. in an amount of about 50 mg to about 300 mg per dose wherein each dose is administered twice a day,
Letrozole p.o. in an amount of about 1.0 to about 10 mg per dose wherein each dose is given once a day, and
Tamoxifen p.o. in an amount of about 10 to about 100 mg per dose wherein each dose is given once a day.

In another embodiment of this invention breast cancer is treated (or prevented) in a patient in need of such treatment wherein said treatment comprises administering to said patient:
the FTI p.o. in an amount of about 100 to 200 mg per dose, wherein each dose is administered twice a day,
Letrozole p.o. in an amount of about 2.5 mg per dose wherein each dose is given once a day, and
Tamoxifen p.o. in an amount of about 20 mg per dose wherein each dose is given once a day.

In another embodiment of this invention breast cancer is treated (or prevented) in a patient in need of such treatment wherein said treatment comprises administering to said patient:
the FTI p.o. in an amount of about 50 mg to about 300 mg per dose wherein each dose is administered twice a day,
Exemestane p.o. in an amount of about 10 to about 50 mg per dose wherein each dose is given once a day, and
Tamoxifen p.o. in an amount of about 10 to about 100 mg per dose wherein each dose is given once a day.

In another embodiment of this invention breast cancer is treated (or prevented) in a patient in need of such treatment wherein said treatment comprises administering to said patient:
the FTI p.o. in an amount of about 100 to 200 mg per dose, wherein each dose is administered twice a day,
Exemestane p.o. in an amount of about 25 mg per dose wherein each dose is given once a day, and
Tamoxifen p.o. in an amount of about 20 mg per dose wherein each dose is given once a day.

Those skilled in the art will appreciate that when other combinations of antihormonal agents are used, the individual antihormonal agent is used in the amounts specified above for that individual antihormonal agent.

Other embodiments of this invention are directed to the methods of treatment described above wherein the FTI is dosed twice a day in an amount of about 100 mg per dose.

Other embodiments of this invention are directed to the methods of treatment described above wherein the FTI is dosed twice a day in an amount of about 200 mg per dose.

Other embodiments of this invention are directed to the methods of treatment described above wherein a chemotherapeutic agent is administered in addition to the FTI and antihormonal agent (or antihormaonal agents). In these embodiments the dosage ranges of the FTI and antihormonal agents are as those described above in the combination therapies, or those described above for the individual FTI and antihormonal agents, and the dosages of the chemotherapeutic agents are those described above for the individual chemotherapeutic agent. The dosages for the chemotherapeutic agents are well known in the art.

Other embodiments of this invention are directed to pharmaceutical compositions comprising the FTI and at least one antihormonal agent and a pharmaceutically acceptable carrier.

Other embodiments of this invention are directed to pharmaceutical compositions comprising the FTI, at least one antihormonal agent, at least one chemotherapeutic agent, and a pharmaceutically acceptable carrier.

Other embodiments of this invention are directed to pharmaceutical compositions comprising the FTI, at least one chemotherapeutic agent, and a pharmaceutically acceptable carrier.

Those skilled in the art will recognize that the actual dosages and protocols for administration employed in the methods of this invention may be varied according to the judgment of the skilled clinician. A determination to vary the dosages and protocols for administration may be made after the skilled clinician takes into account such factors as the patient's age, condition and size, as well as the severity of the cancer being treated and the response of the patient to the treatment.

The particular choice of antihormonal agents, optional chemotherapeutic agents and optional radiation will depend upon the diagnosis of the attending physicians and their judgment of the condition of the patient and the appropriate treatment protocol.

The determination of the order of administration, and the number of repetitions of administration of the antihormonal agents, optional chemotherapeutic agents and optional radiation during a treatment protocol, is well within the knowledge of the skilled physician after evaluation of the breast cancer being treated and the condition of the patient.

Thus, in accordance with experience and knowledge, the practicing physician can modify each protocol for the administration of antihormonal agents, optional chemotherapeutic agents and optional radiation according to the individual patient's needs, as the treatment proceeds. All such modifications are within the scope of the present invention.

The attending clinician, in judging whether treatment is effective at the dosage administered, will consider the general well-being of the patient as well as more definite signs such as relief of cancer-related symptoms (e.g., pain), inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

Assay Procedure

Cell lines. MCF-7 cells were provided from Dr. A. Brodie (University of Maryland School of Medicine, Baltimore, Md.) and cultured in DMEM supplemented with 5% fetal bovine serum (FBS) and 1% penicillin/streptomycin (P/S). MCF-7arom cells were provided from Dr. S. Chen (Beckman Research Institute of the City of Hope, Duarte, Calif.) and cultured in DMEM supplemented with 5% FBS and 750 μg/ml geneticin.

Growth studies in vitro. Six days prior to plating in 96-well plates, MCF-7 cells growing in T-75 flasks (30-50% confluent) were washed extensively with phenyl red-free DPBS and transferred to estrogen-depleted medium. Estrogen-depleted medium was phenyl red-free DMEM/F-12 containing 10% heat-treated and dextran-coated charcoal-treated fetal bovine serum and 1% penicillin/streptomycin solution. Estrogen depleted media was refreshed 3 days prior to plating. On Day 0, cells were seeded (1,200 cells/well) into 96-well plates in estrogen-depleted medium and allowed to attach. On Day 1 the media was aspirated and replaced in 6-well replicates with estrogen-depleted medium supplemented with E2 (1 nM), and 4-OH Tamoxifen (Sigma Chemical Company, St Louis, Mo. in the range 10 nM-10 μM), or Fulvestrant (Tocris, Ellisville, Mo. in the range 10 nM-1.0 μM), FTI (in the range 10 nM-10 μM), and a combination of the antiestrogens and FTI. Media containing drugs was refreshed on Day 3. The effects of treatment on cell proliferation were determined on Day 6 using the CellTiter-Glo luminescent viability assay (Promega Corp., Madison, Wis.).

Growth Studies in vivo. Female ovariectomized athymic nude mice were obtained from Charles River Laboratories (Worcester, Mass.). Androstenedione (Δ4A) pellets (25 mg and 15 mg, 90-day slow release) and 17β-estradiol (E2) pellets (0.72 mg, 60-day slow release) were from Innovative Research of America (Saratoga, Fla.). Anastrozole and Letrozole were obtained from Sequoia Research Products, Oxford, United Kingdom) and Tamoxifen was from Sigma Chemical Company.

MCF-7arom breast tumor growth was performed as described previously (Lu et al., 1999, Breast Cancer Res. Treat., 57, 183-192; Long et al., 2002, Clin Cancer Res., 8, 2378-2388) with minor modifications. To determine the effect of combined FTI plus Anastrozole on the growth of MCF-7arom breast tumor xenografts, 2.5×10⁶ MCF-7arom cells were inoculated into the right flank of the animals in 100 μl of Matrigel (BD Biosciences, Bedford, Mass.). Animals had been implanted with a 25 mg, 90-day Δ4A pellet the previous day. Fourteen days after cell inoculation, the animals were grouped (n=10) for treatment with:
1) Vehicle
2) Anastrozole (5 mg per kg [mpk], orally [p.o.], twice a day [b.i.d.])
3) FTI (20 mpk, p.o., b.i.d.)
4) FTI (40 mpk, p.o., b.i.d.)
5) FTI (60 mpk, p.o., b.i.d.)
6) FTI (20 mpk, p.o., b.i.d.)+Anastrozole (5 mpk, p.o., b.i.d.)
7) FTI (40 mpk, p.o., b.i.d.)+Anastrozole (5 mpk, p.o., b.i.d.)
8) FTI (60 mpk, p.o., b.i.d.)+Anastrozole (5 mpk, p.o., b.i.d.)

Treatment was continued for 28 days and tumor volumes were measured twice a week with calipers. Tumor volumes and animal body weights were recorded using LABCAT (Innovative Programming Associates Inc., Princeton, N.J.). Tumor volumes were calculated by the formula (w×l×h)/2.

To determine the effect of combined FTI plus Letrozole on the growth of MCF-7arom breast tumor xenografts, 5×10⁶ MCF-7arom cells were inoculated into the right flank of the animals in 100 μl of Matrigel. Animals had been implanted with a 15 mg, 90-day Δ4A pellet the previous day. Fourteen days after cell inoculation, the animals were grouped (n=10) for treatment with:
1) Vehicle
2) Letrozole (2.5 mpk, p.o., once a day [q.d.])
3) FTI (20 mpk, p.o., b.i.d.)
4) FTI (40 mpk, p.o., b.i.d.)
5) FTI (20 mpk, p.o., b.i.d.)+Letrozole (2.5 mpk, p.o., q.d.)
6) FTI (40 mpk, p.o., b.i.d.)+Letrozole (2.5 mpk, p.o., q.d.)

Treatment was continued for 28 days and tumor volumes were measured twice a week with calipers as described above.

To determine the effect of combined FTI plus Tamoxifen on the growth of MCF-7arom breast tumor xenografts, 5×10⁶ MCF-7arom cells were inoculated into the right flank of the animals in 100 μl of Matrigel. Animals had been implanted with a 15 mg, 90-day Δ4A pellet the previous day. Fourteen days after cell inoculation, the animals were grouped (n=10) for treatment with:
1) Vehicle
2) Tamoxifen (25 mpk, p.o., q.d.)
3) FTI (20 mpk, p.o., b.i.d.)
4) FTI (40 mpk, p.o., b.i.d.)
5) FTI (20 mpk, p.o., b.i.d.)+Tamoxifen (25 mpk, p.o., q.d.)
6) FTI (40 mpk, p.o., b.i.d.)+Tamoxifen (25 mpk, p.o., q.d.)

Treatment was continued for 28 days and tumor volumes were measured twice a week with calipers as described above.

MCF-7 breast tumor growth was performed as described previously (Osborne et al., 1995, J. Natl Cancer Inst., 87, 746-750) with minor modifications. To determine the effect of combined FTI plus Tamoxifen on the growth of MCF-7 breast tumor xenografts, 5.0×10⁶ MCF-7 cells were inoculated into the right flank of the animals in 100 μl of Matrigel. Animals had been implanted with a 0.72 mg, 60-day pellet the previous day. Fourteen days after cell inoculation, the animals were grouped for treatment with:
1) Vehicle
2) Tamoxifen (25 mpk, p.o., q.d.)
3) FTI (20 mpk, p.o., b.i.d.)
4) FTI (40 mpk, p.o., b.i.d.)
5) FTI (20 mpk, p.o., b.i.d.)+Tamoxifen (25 mpk, p.o., q.d.)
6) FTI (40 mpk, p.o., b.i.d.)+Tamoxifen (25 mpk, p.o., q.d.)

Treatment was continued for 28 days and tumor volumes were measured twice a week with calipers as described above.

Results

MCF-7 cell growth in vitro. MCF-7 cells were sensitive to treatment with single-agent 4-OH Tamoxifen (inhibited cell growth with an IC₅₀ value of 0.45 μM) and FTI (IC₅₀ value of 0.04 μM) and both drugs inhibited cell proliferation in a dose-dependent manner (FIG. 1). The combination of FTI plus 4-OH Tamoxifen was more effective at inhibiting MCF-7 cell proliferation than treatment with either drug alone (FIG. 1). For example, single-agent 4-OH Tamoxifen (1.0 μM) inhibited cell proliferation by 55% and single-agent FTI (0.1 μM) inhibited cell proliferation by 56%. When the 2 drugs were combined, MCF-7 cell proliferation was inhibited by 75%. At each of the drug concentrations tested the combination of FTI plus 4-OH Tamoxifen was superior to single-agent 4-OH Tamoxifen at inhibiting cell proliferation. This increased efficacy was also observed when FTI was combined with the pure antiestrogen Fulvestrant (FIG. 2). For example, single-agent Fulvestrant (0.1 μM) inhibited cell proliferation by 53% and single-agent FTI (0.1 μM) inhibited cell proliferation by 43%. However, the combination of FTI plus Fulvestrant at the same concentrations inhibited cell proliferation by 72%. These results clearly demonstrate that the combination of FTI plus antiestrogens is superior to single-agent treatment with either drug alone at inhibiting the proliferation of hormone-dependent MCF-7 human breast cancer cells.

MCF-7arom tumor growth in vivo. In the first experiment the effects of combining FTI (20, 40, and 60 mpk, b.i.d.) and the aromatase inhibitor Anastrozole (5 mpk, b.i.d.) on the growth of MCF-7arom human breast tumors was determined (FIGS. 3-6). Compared to the vehicle-treated animals, single-agent Anastrozole inhibited tumor growth by 62% over the 28 days of treatment, but did not induce tumor regression (FIG. 3). Single-agent FTI (20 mpk) inhibited tumor growth by 56% and, again, did not induce tumor regression. However, when Anastrozole and FTI (20 mpk) were combined, tumor growth was inhibited by 130% and tumors regressed to 69% of their initial (untreated) starting volume (31% regression, FIG. 3). Single-agent FTI inhibited MCF-7arom tumor growth in a dose-dependent manner and at doses of 40 mpk and 60 mpk tumors regressed by 16% and 40% respectively. Regardless, when treated with the combinations of FTI (40 mpk and 60 mpk) plus Anastrozole MCF-7arom tumors regressed by 67% and 70% of their initial starting volume, respectively, indicating the superior anti-tumor efficacy of the combination. In fact, at each of the doses of FTI tested the combination of FTI plus Anastrozole was significantly better than either single-agent Anastrozole or single-agent FTI (FIG. 6).

In the second experiment the effect of combining FTI (40 mpk, b.i.d.) and the aromatase inhibitor Letrozole (2.5 mpk, q.d.) on the growth of MCF-7arom breast tumor xenografts was determined (FIG. 7). By Day 10 of treatment, single-agent Letrozole had inhibited MCF-7arom tumor growth by 127% (25% regression) and single-agent FTI had inhibited tumor growth by 16%. However, the combination of FTI plus Letrozole inhibited tumor growth by 152% and tumors had regressed to 50% of their untreated starting volume.

In the same experiment, the effect of combining FTI (40 mpk, b.i.d.) and Tamoxifen (25 mpk, q.d.) was also determined (FIG. 8). By Day 10 of treatment, single-agent Tamoxifen had inhibited MCF-7arom tumor growth by only 22% and single-agent FTI had inhibited tumor growth by only 16%. However, the combination of FTI plus Tamoxifen had inhibited tumor growth by 116% and tumors had regressed by 17% to 83% of their untreated starting volume. The induction of tumor regression by the combination of FTI plus Tamoxifen clearly demonstrates that combined therapy is superior to single-agent therapy with either drug alone and that the two drugs are likely to be inhibiting tumor growth in a synergistic manner.

In the final experiment the effect of combining FTI (40 mpk, b.i.d.) and Tamoxifen (25 mpk, q.d.) on the growth of estrogen-stimulated MCF-7 human breast cancer xenografts was determined. MCF-7 cells are sensitive to the antiproliferative effects of single-agent FTI in vitro (FIGS. 1 and 2). In the animals, MCF-7 tumor xenografts were also sensitive to treatment with single agent FTI and by Day 14 of treatment tumors had regressed by 57% of their initial starting volume. Single-agent Tamoxifen inhibited tumor growth by only 29% over the 14 days of treatment. However, the combination of FTI plus Tamoxifen was not better than single-agent FTI at inhibiting MCF-7 tumor growth because single-agent FTI was very effective on its own. Because MCF-7 tumor xenografts in this protocol were very sensitive to treatment with single-agent FTI the results obtained are not conclusive of the effect that would be obtained from the combination of FTI and Tamoxifen. It is believed that a protocol using a lower dose of FTI may demonstrate an advantage to the combination of the lower dose of FTI with Tamoxifen.

### Conclusions

The combination of FTI plus 4-OH Tamoxifen is superior to single-agent treatment with either drug alone at inhibiting the proliferation of MCF-7 cells in vitro.

The combination of FTI plus Fulvestrant is superior to single-agent treatment with either drug alone at inhibiting the proliferation of MCF-7 cells in vitro.

The combination of FTI plus Anastrozole is superior to single-agent treatment with either drug alone at inhibiting the proliferation of MCF-7arom human breast tumor xenografts in vivo. Moreover, in contrast to the results observed with low doses of single-agent FTI and Anastrozole, the combination of FTI plus Anastrozole induces marked tumor regression.

The combination of FTI plus Letrozole is superior to single-agent treatment with either drug alone at inhibiting the proliferation of MCF-7arom human breast tumor xenografts in vivo. Moreover, although single-agent Letrozole induces regression of MCF-7arom breast tumor xenografts, the combination of FTI plus Letrozole is more effective at inducing tumor regression.

The combination of FTI plus Tamoxifen is superior to single-agent treatment with either drug alone at inhibiting the proliferation of MCF-7arom human breast tumor xenografts in vivo. Moreover, in contrast to the results observed with single-agent FTI and Tamoxifen, the combination of FTI plus Tamoxifen induces marked tumor regression.

The effects of combining FTI and Tamoxifen on the growth of MCF-7 human breast tumor xenografts remain to be determined.

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

1. A method of treating breast cancer in a patient in need of such treatment comprising administering to said patient a therapeutically effective amount of the farnesyl transferase inhibitor: 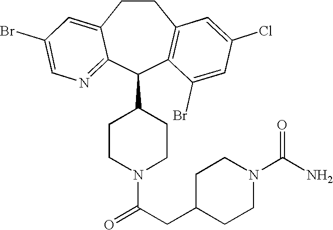
and a therapeutically effective amount of at least one antihormonal agent selected from the group consisting of:
(a) aromatase inhibitors;
(b) antiestrogens; and
(c) LHRH analogues; and
said treatment optionally including the administration of at least one chemotherapeutic agent.

2. The method of claim 1 wherein said treatment comprises the administration of said farnesyl transferase inhibitor and at least one antihormonal agent selected from the group consisting of:
(a) aromatase inhibitors;
(b) antiestrogens; and
(c) LHRH analogues.

3. The method of claim 1 wherein said treatment comprises the administration of said farnesyl transferase inhibitor and at least one aromatase inhibitor.

4. The method of claim 2 wherein said treatment comprises the administration of said farnesyl transferase inhibitor and at least one aromatase inhibitor.

5. The method of claim 1 wherein said treatment comprises the administration of said farnesyl transferase inhibitor and at least one antiestrogen.

6. The method of claim 2 wherein said treatment comprises the administration of said farnesyl transferase inhibitor and at least one antiestrogen.

7. The method of claim 1 wherein said treatment comprises the administration of said farnesyl transferase inhibitor and at least one aromatase inhibitor and at least one antiestrogen.

8. The method of claim 2 wherein said treatment comprises the administration of said farnesyl transferase inhibitor and at least one aromatase inhibitor and at least one antiestrogen.

9. The method of claim 1 wherein said treatment comprises the administration of said farnesyl transferase inhibitor, at least one aromatase inhibitor, and at least one chemotherapeutic agent.

10. The method of claim 1 wherein said treatment comprises the administration of said farnesyl transferase inhibitor, at least one antiestrogen, and at least one chemotherapeutic agent.

11. The method of claim 1 wherein said treatment comprises the administration of said farnesyl transferase inhibitor, at least one aromatase inhibitor, at least one antiestrogen, and at least one chemotherapeutic agent.

12. The method of claim 1 wherein said
(a) aromatase inhibitors are selected from the group consisting of: Anastrozole, Letrozole, Exemestane, Fadrozole and Formestane;
(b) antiestrogens are selected from the group consisting of: Tamoxifen, Fulvestrant, Raloxifene, and Acolbifene;
(c) LHRH analogues are selected from the group consisting of: Goserelin and Leuproelin; and
(d) chemotherapeutic agents are selected from the group consisting of: Trastuzumab, Gefitinib, Erlotinib, Bevacizumab, Cetuximab, and Bortezomib.

13. The method of claim 2 wherein said
(a) aromatase inhibitors are selected from the group consisting of: Anastrozole, Letrozole, Exemestane, Fadrozole and Formestane;
(b) antiestrogens are selected from the group consisting of: Tamoxifen, Fulvestrant, Raloxifene, and Acolbifene; and
(c) LHRH analogues are selected from the group consisting of: Goserelin and Leuproelin.

14. The method of claim 1 wherein said treatment comprises the administration of said farnesyl transferase inhibitor and Anastrozole.

15. The method of claim 1 wherein said treatment comprises the administration of said farnesyl transferase inhibitor and Letrozole.

16. The method of claim 1 wherein said treatment comprises the administration of said farnesyl transferase inhibitor and Exemestane.

17. The method of claim 1 wherein said treatment comprises the administration of said farnesyl transferase inhibitor and Fadrozole.

18. The method of claim 1 wherein said treatment comprises the administration of said farnesyl transferase inhibitor and Formestane.

19. The method of claim 1 wherein said treatment comprises the administration of said farnesyl transferase inhibitor and Tamoxifen.

20. The method of claim 1 wherein said treatment comprises the administration of said farnesyl transferase inhibitor and Fulvestrant.

21. The method of claim 1 wherein said treatment comprises the administration of said farnesyl transferase inhibitor and Raloxifene.

22. The method of claim 1 wherein said treatment comprises the administration of said farnesyl transferase inhibitor and Acolbifene.

23. The method of claim 1 wherein said treatment comprises the administration of said farnesyl transferase inhibitor and Goserelin.

24. The method of claim 1 wherein said treatment comprises the administration of said farnesyl transferase inhibitor and Leuprolein.

25. The method of claim 1 wherein said treatment comprises the administration of said farnesyl transferase inhibitor, Anastrozole, and an antiestrogen selected from the group consisting of: Tamoxifen, Fulvestrant, Raloxifene, and Acolbifene.

26. The method of claim 1 wherein said treatment comprises the administration of said farnesyl transferase inhibitor, Letrozole, and an antiestrogen selected from the group consisting of: Tamoxifen, Fulvestrant, Raloxifene, and Acolbifene.

27. The method of claim 1 wherein said treatment comprises the administration of said farnesyl transferase inhibitor, Exemestane, and an antiestrogen selected from the group consisting of: Tamoxifen, Fulvestrant, Raloxifene, and Acolbifene.

28. The method of claim 1 wherein said treatment comprises the administration of said farnesyl transferase inhibitor, Fadrozole, and an antiestrogen selected from the group consisting of: Tamoxifen, Fulvestrant, Raloxifene, and Acolbifene.

29. The method of claim 1 wherein said treatment comprises the administration of said farnesyl transferase inhibitor, Formestane, and an antiestrogen selected from the group consisting of: Tamoxifen, Fulvestrant, Raloxifene, and Acolbifene.

30. The method of claim 1 wherein said treatment comprises the administration of said farnesyl transferase inhibitor, Anastrozole, and Tamoxifen.

31. The method of claim 1 wherein said treatment comprises the administration of said farnesyl transferase inhibitor, Letrozole, and Tamoxifen.

32. The method of claim 1 wherein said treatment comprises the administration of said farnesyl transferase inhibitor, Exemestane, and Tamoxifen.

33. The method of claim 1 wherein said treatment comprises the administration of said farnesyl transferase inhibitor, Fadrozole, and Tamoxifen.

34. The method of claim 1 wherein said treatment comprises the administration of said farnesyl transferase inhibitor, Formestane, and Tamoxifen.

35. The method of claim 1 wherein said treatment comprises the administration of said farnesyl transferase inhibitor, Anastrozole, and Fulvestrant.

36. The method of claim 1 wherein said treatment comprises the administration of said farnesyl transferase inhibitor, Letrozole, and Fulvestrant.

37. The method of claim 1 wherein said treatment comprises the administration of said farnesyl transferase inhibitor Exemestane, and Fulvestrant.

38. The method of claim 1 wherein said treatment comprises the administration of said farnesyl transferase inhibitor, Fadrozole, and Fulvestrant.

39. The method of claim 1 wherein said treatment comprises the administration of said farnesyl transferase inhibitor, Formestane, and Fulvestrant.

40. The method of claim 1 wherein said treatment comprises the administration of said farnesyl transferase inhibitor, Anastrozole, and a chemotherapeutic agent selected from the group consisting of: Trastuzumab, Gefitinib, Erlotinib, Bevacizumab, Cetuximab, and Bortezomib.

41. The method of claim 1 wherein said treatment comprises the administration of said farnesyl transferase inhibitor, Letrozole, and a chemotherapeutic agent selected from the group consisting of: Trastuzumab, Gefitinib, Erlotinib, Bevacizumab, Cetuximab, and Bortezomib.

42. The method of claim 1 wherein said treatment comprises the administration of said farnesyl transferase inhibitor, Exemestane, and a chemotherapeutic agent selected from the group consisting of: Trastuzumab, Gefitinib, Erlotinib, Bevacizumab, Cetuximab, and Bortezomib.

43. The method of claim 1 wherein said treatment comprises the administration of said farnesyl transferase inhibitor, Fadrozole, and a chemotherapeutic agent selected from the group consisting of: Trastuzumab, Gefitinib, Erlotinib, Bevacizumab, Cetuximab, and Bortezomib.

44. The method of claim 1 wherein said treatment comprises the administration of said farnesyl transferase inhibitor, Formestane, and a chemotherapeutic agent selected from the group consisting of: Trastuzumab, Gefitinib, Erlotinib, Bevacizumab, Cetuximab, and Bortezomib.

45. The method of claim 1 wherein said treatment comprises the administration of said farnesyl transferase inhibitor, Tamoxifen, and a chemotherapeutic agent selected from the group consisting of: Trastuzumab, Gefitinib, Erlotinib, Bevacizumab, Cetuximab, and Bortezomib.

46. The method of claim 1 wherein said treatment comprises the administration of said farnesyl transferase inhibitor, Fulvestrant, and a chemotherapeutic agent selected from the group consisting of: Trastuzumab, Gefitinib, Erlotinib, Bevacizumab, Cetuximab, and Bortezomib.

47. The method of claim 1 wherein said treatment comprises the administration of said farnesyl transferase inhibitor, Raloxifene, and a chemotherapeutic agent selected from the group consisting of: Trastuzumab, Gefitinib, Erlotinib, Bevacizumab, Cetuximab, and Bortezomib.

48. The method of claim 1 wherein said treatment comprises the administration of said farnesyl transferase inhibitor, Acolbifene, and a chemotherapeutic agent selected from the group consisting of: Trastuzumab, Gefitinib, Erlotinib, Bevacizumab, Cetuximab, and Bortezomib.

49. The method of claim 1 wherein said treatment comprises the administration of said farnesyl transferase inhibitor, Goserelin, and a chemotherapeutic agent selected from the group consisting of: Trastuzumab, Gefitinib, Erlotinib, Bevacizumab, Cetuximab, and Bortezomib.

50. The method of claim 1 wherein said treatment comprises the administration of said farnesyl transferase inhibitor, Leuprolein, and a chemotherapeutic agent selected from the group consisting of: Trastuzumab, Gefitinib, Erlotinib, Bevacizumab, Cetuximab, and Bortezomib.

51. The method of claim 1 wherein said treatment comprises the administration of said farnesyl transferase inhibitor, Anastrozole, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

52. The method of claim 1 wherein said treatment comprises the administration of said farnesyl transferase inhibitor, Letrozole, an antiestrogen selected from the group consisting of: Tamoxifen, Fulvestrant, Raloxifene, and Acolbifene, and a chemotherapeutic agent selected from the group consisting of:
Trastuzumab, Gefitinib, Erlotinib, Bevacizumab, Cetuximab, and Bortezomib.

53. The method of claim 1 wherein said treatment comprises the administration of said farnesyl transferase inhibitor, Exemestane, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

54. The method of claim 1 wherein said treatment comprises the administration of said farnesyl transferase inhibitor, Fadrozole, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

55. The method of claim 1 wherein said treatment comprises the administration of said farnesyl transferase inhibitor, Formestane, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

56. The method of claim 1 wherein said treatment comprises the administration of said farnesyl transferase inhibitor, Anastrozole, Tamoxifen, and a chemotherapeutic agent selected from the group consisting of: Trastuzumab, Gefitinib, Erlotinib, Bevacizumab, Cetuximab, and Bortezomib.

57. The method of claim 1 wherein said treatment comprises the administration of said farnesyl transferase inhibitor, Letrozole, Tamoxifen, and a chemotherapeutic agent selected from the group consisting of: Trastuzumab, Gefitinib, Erlotinib, Bevacizumab, Cetuximab, and Bortezomib.

58. The method of claim 1 wherein said treatment comprises the administration of said farnesyl transferase inhibitor, Exemestane, Tamoxifen, and a chemotherapeutic agent selected from the group consisting of: Trastuzumab, Gefitinib, Erlotinib, Bevacizumab, Cetuximab, and Bortezomib.

59. The method of claim 1 wherein said treatment comprises the administration of said farnesyl transferase inhibitor, Fadrozole, Tamoxifen, and a chemotherapeutic agent selected from the group consisting of: Trastuzumab, Gefitinib, Erlotinib, Bevacizumab, Cetuximab, and Bortezomib.

60. The method of claim 1 wherein said treatment comprises the administration of said farnesyl transferase inhibitor, Formestane, Tamoxifen, and a chemotherapeutic agent selected from the group consisting of: Trastuzumab, Gefitinib, Erlotinib, Bevacizumab, Cetuximab, and Bortezomib.

61. The method of claim 1 wherein said treatment comprises the administration of said farnesyl transferase inhibitor, Anastrozole, Fulvestrant, and a chemotherapeutic agent selected from the group consisting of: Trastuzumab, Gefitinib, Erlotinib, Bevacizumab, Cetuximab, and Bortezomib.

62. The method of claim 1 wherein said treatment comprises the administration of said farnesyl transferase inhibitor, Letrozole, Fulvestrant, and a chemotherapeutic agent selected from the group consisting of: Trastuzumab, Gefitinib, Erlotinib, Bevacizumab, Cetuximab, and Bortezomib.

63. The method of claim 1 wherein said treatment comprises the administration of said farnesyl transferase inhibitor Exemestane, Fulvestrant, and a chemotherapeutic agent selected from the group consisting of: Trastuzumab, Gefitinib, Erlotinib, Bevacizumab, Cetuximab, and Bortezomib.

64. The method of claim 1 wherein said treatment comprises the administration of said farnesyl transferase inhibitor, Fadrozole, Fulvestrant, and a chemotherapeutic agent selected from the group consisting of: Trastuzumab, Gefitinib, Erlotinib, Bevacizumab, Cetuximab, and Bortezomib.

65. The method of claim 1 wherein said treatment comprises the administration of said farnesyl transferase inhibitor, Formestane, Fulvestrant, and a chemotherapeutic agent selected from the group consisting of: Trastuzumab, Gefitinib, Erlotinib, Bevacizumab, Cetuximab, and Bortezomib.

66. The method of claim 1 wherein said treatment comprises administering a therapeutically effective amount of:
(a) the FTI
(b) at least one aromatase inhibitor; and
(c) at least one LHRH analogue.

67. The method of claim 1 wherein said treatment comprises administering a therapeutically effective amount of:
(a) the FTI
(b) at least one antiestrogen; and
(c) at least one LHRH analogue.

68. The method of claim 1 wherein said treatment comprises administering a therapeutically effective amount of:
(a) the FTI
(b) at least one aromatase inhibitor that is selected from the group consisting of Anastrozole, Letrozole, Exemestane, Fadrozole and Formestane; and
(c) at least one LHRH analogue that is selected from the group consisting of: Goserelin and Leuprolide.

69. The method of claim 1 wherein said treatment comprises administering a therapeutically effective amount of:
(a) the FTI
(b) at least one antiestrogen that is selected from the group consisting of: Tamoxifen, Fulvestrant, Raloxifene, and Acolbifene; and
(c) at least one LHRH analogue that is selected from the group consisting of: Goserelin and Leuprolide.

70. A pharmaceutical composition comprising the FTI of claim 1, at least one antihormonal agent and a pharmaceutically acceptable carrier.

71. A pharmaceutical composition comprising the FTI of claim 1, at least one antihormonal agent, at least one chemotherapeutic agent, and a pharmaceutically acceptable carrier.

72. A pharmaceutical composition comprising the FTI of claim 1, at least one chemotherapeutic agent, and a pharmaceutically acceptable carrier.
